(19) United States
(12) Patent Application Publication
(10) Pub. No.: US 2017/0259066 A1
(43) Pub. Date: Sep. 14, 2017

(54) SYSTEMS AND METHODS FOR DISRUPTING NEURONAL OSCILLATION USING DEEP BRAIN STIMULATION

(71) Applicant: Pacesetter, Inc., Sunnyvale, CA (US)

(72) Inventors: Edward Karst, Los Angeles, CA (US); Alexander Kent, Mountain View, CA (US); Lalit Venkatesan, Prosper, TX (US); Timothy A. Fayram, Gilroy, CA (US)

(21) Appl. No.: 15/063,880

(22) Filed: Mar. 8, 2016

(51) Int. Cl. A61N 1/36; A61N 1/378; A61N 1/372; A61N 1/05

(52) U.S. Cl. CPC A61N 1/36178; A61N 1/0534; A61N 1/36067; A61N 1/3787; A61N 1/37264

(57) ABSTRACT

The present disclosure provides systems and methods for disrupting neuronal oscillations. A neurostimulation system includes a stimulation lead comprising at least one contact, and an implantable pulse generator (IPG) communicatively coupled to the stimulation lead and configured to cause stimulation to be applied to a patient using no more than two contacts of the stimulation lead by causing a first burst of stimulation to be delivered, and causing a second burst of stimulation to be delivered within a neuronal refractory period that follows the first burst of stimulation.

### FIELD OF THE DISCLOSURE

The present disclosure relates generally to neurostimulation systems, and more particularly to disrupting neuronal oscillations in a patient using deep brain stimulation.

### BACKGROUND ART

Parkinson's disease (PD) is a neurogenerative disorder that is characterized by pathological rhythmic neuronal oscillations that lead to symptoms such as tremor and bradykinesia. Deep brain stimulation (DBS) systems may be used to apply stimulation to provide symptom relief. For example, for PD, stimulation may be applied to a region in the basal ganglia, such as the subthalamic nucleus or globus pallidus internus. In at least some known DBS devices, stimulation is applied using a train of individual pulses at a fixed frequency and pulse width.

At least some other known DBS devices apply stimulation in accordance with a coordinated reset stimulation pattern. For a coordinated reset, bursts of pulses are delivered by three different electrodes (i.e., contacts) in a random or pseudo-random sequence. This pattern activates three different populations of neurons within or proximate to the targeted small nucleus near each of the stimulating electrodes, and breaks the coordinated oscillation characteristic of PD. The symptom relief provided by a coordinated reset may be sustained for hours, or even days after discontinuing stimulation.

However, it is not always possible to apply stimulation using three different electrodes that target the subthalamic nucleus for symptom relief without side effects. Further, it may be advantageous to use fewer electrodes while targeting the exact site of maximum symptom relief.

### BRIEF SUMMARY OF THE DISCLOSURE

In one embodiment, the present disclosure is directed to a neurostimulation system for disrupting neuronal oscillations. The neurostimulation system includes a stimulation lead comprising at least one contact, and an implantable pulse generator (IPG) communicatively coupled to the stimulation lead and configured to cause stimulation to be applied to a patient using no more than two contacts of the stimulation lead by causing a first burst of stimulation to be delivered, and causing a second burst of stimulation to be delivered within a neuronal refractory period that follows the first burst of stimulation.

In another embodiment, the present disclosure is directed to an implantable pulse generator (IPG) for use with a neurostimulation system for disrupting neuronal oscillations. The IPG includes a memory device, and a controller communicatively coupled to the memory device, the controller configured to cause stimulation to be applied to a patient using no more than two contacts of a stimulation lead coupled to the IPG by causing a first burst of stimulation to be delivered, and causing a second burst of stimulation to be delivered within a neuronal refractory period that follows the first burst of stimulation.

In another embodiment, the present disclosure is directed to a method of applying stimulation using no more than two contacts of a stimulation lead to disrupt neuronal oscillations in a patient. The method includes delivering a first burst of stimulation using the stimulation lead, and delivering a second burst of stimulation within a neuronal refractory period that follows the first burst of stimulation.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic view of one embodiment of a stimulation system.

FIG. 2 us a schematic view of one embodiment of a portion of a stimulation lead that may be used with the stimulation system shown in FIG. 1.

FIG. 3 is a diagram of an example stimulation pattern than may be applied using the stimulation lead shown in FIG. 2.

FIG. 4 is a diagram of an example stimulation pattern than may be applied using the stimulation lead shown in FIG. 2.

FIG. 5 is a perspective view of one embodiment of a stimulation lead that may be used with the stimulation system shown in FIG. 1.

FIG. 6 is a flow chart of one embodiment of a method for applying stimulation to disrupt neuronal oscillations in a patient.

FIG. 7 is a flow chart of one embodiment of a method for delivering first and second bursts of stimulation to disrupt neuronal oscillations in a patient.

FIG. 8 is a flow chart of another embodiment of a method for delivering first and second bursts of stimulation to disrupt neuronal oscillations in a patient.

FIG. 9 is a flow chart of another embodiment of a method for delivering first and second bursts of stimulation to disrupt neuronal oscillations in a patient.

FIG. 10 is a flow chart of another embodiment of a method for delivering first and second bursts of stimulation to disrupt neuronal oscillations in a patient.

FIG. 11 is a flow chart of another embodiment of a method for delivering first and second bursts of stimulation to disrupt neuronal oscillations in a patient.

Corresponding reference characters indicate corresponding parts throughout the several views of the drawings.

### DETAILED DESCRIPTION OF THE DISCLOSURE

The present disclosure provides systems and methods for disrupting the pathological oscillation of Parkinson's disease (PD) using only one or two contacts (i.e., electrodes) of a neurostimulation system. A first burst of stimulation is applied to a patient. Subsequently, and within a neuronal refractory period following the first burst, a second burst of stimulation is applied to the patient. Notably, using the systems and methods described herein, neuronal oscillations may be disrupted using no more than two contacts of a stimulation lead.

Neurostimulation systems are devices that generate electrical pulses and deliver the pulses to nerve tissue of a patient to treat a variety of disorders. One category of neurostimulation systems is deep brain stimulation (DBS). In DBS, electrical pulses are delivered to parts of a subject's brain, for example, for the treatment of movement and effective disorders such as PD and essential tremor.

Neurostimulation systems generally include a pulse generator and one or more leads. A stimulation lead includes a lead body of insulative material that encloses wire conductors. The distal end of the stimulation lead includes multiple electrodes, or contacts, that are electrically coupled to the wire conductors. The proximal end of the lead body includes multiple terminals (also electrically coupled to the wire conductors) that are adapted to receive electrical pulses. In DBS systems, the stimulation lead is implanted within the brain tissue to deliver the electrical pulses. The stimulation leads are then tunneled to another location within the patient's body to be electrically connected with a pulse generator or, alternatively, to an “extension.” The pulse generator is typically implanted within a subcutaneous pocket created during the implantation procedure.

The pulse generator is typically implemented using a metallic housing that encloses circuitry for generating the electrical pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on a stimulation lead.

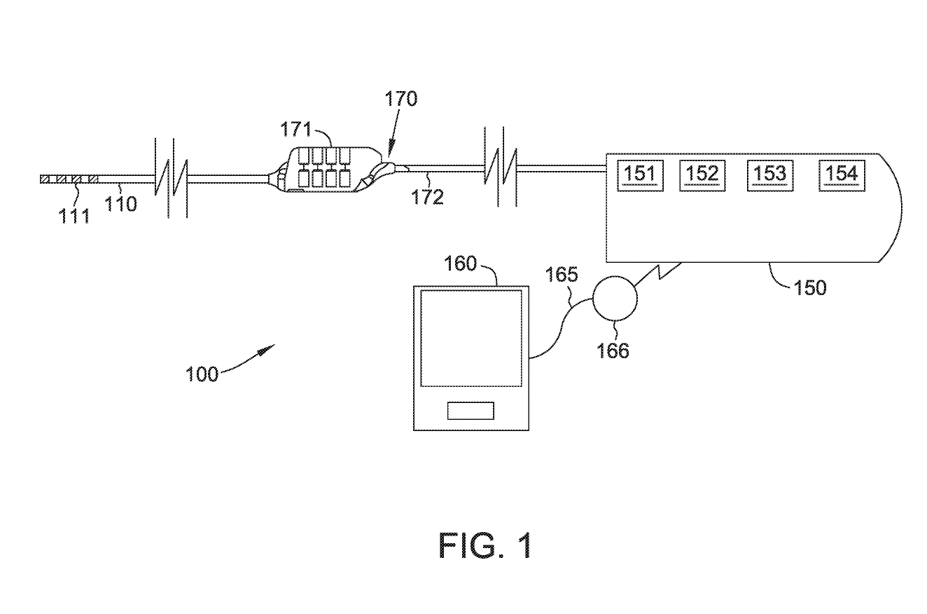

Referring now to the drawings, and in particular to FIG. 1, a stimulation system is indicated generally at 100. Stimulation system 100 generates electrical pulses for application to tissue of a patient, or subject, according to one embodiment. System 100 includes an implantable pulse generator 150 that is adapted to generate electrical pulses for application to tissue of a patient. Implantable pulse generator 150 typically includes a metallic housing that encloses a controller 151, pulse generating circuitry 152, a battery 153, far-field and/or near field communication circuitry 154, and other appropriate circuitry and components of the device. Controller 151 typically includes a microcontroller or other suitable processor for controlling the various other components of the device. Software code is typically stored in memory of pulse generator 150 for execution by the microcontroller or processor to control the various components of the device.

Pulse generator 150 may comprise one or more attached extension components 170 or be connected to one or more separate extension components 170. Alternatively, one or more stimulation leads 110 may be connected directly to pulse generator 150. Within pulse generator 150, electrical pulses are generated by pulse generating circuitry 152 and are provided to switching circuitry. The switching circuit connects to output wires, traces, lines, or the like (not shown) which are, in turn, electrically coupled to internal conductive wires (not shown) of a lead body 172 of extension component 170. The conductive wires, in turn, are electrically coupled to electrical connectors (e.g., “Bal-Seal” connectors) within connector portion 171 of extension component 170. The terminals of one or more stimulation leads 110 are inserted within connector portion 171 for electrical connection with respective connectors. Thereby, the pulses originating from pulse generator 150 and conducted through the conductors of lead body 172 are provided to stimulation lead 110. The pulses are then conducted through the conductors of lead 110 and applied to tissue of a patient via electrodes 111. Any suitable known or later developed design may be employed for connector portion 171.

For implementation of the components within pulse generator 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 2006/0170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within pulse generator 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number and type of electrodes 111, terminals, and internal conductors.

Controller device 160 may be implemented to recharge battery 153 of pulse generator 150 (although a separate recharging device could alternatively be employed). A “wand” 165 may be electrically connected to controller device through suitable electrical connectors (not shown). The electrical connectors are electrically connected to coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The patient then places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. Controller device 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in secondary coil. The current induced in the coil of the implantable pulse generator is rectified and regulated to recharge battery of generator 150. The charging circuitry may also communicate status messages to controller device 160 during charging operations using pulse-loading or any other suitable technique. For example, controller device 160 may communicate the coupling status, charging status, charge completion status, etc.

External controller device 160 is also a device that permits the operations of pulse generator 150 to be controlled by user after pulse generator 150 is implanted within a patient, although in alternative embodiments separate devices are employed for charging and programming. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package or provided as a separate attachable device. The interface functionality of controller device 160 is implemented using suitable software code for interacting with the user and using the wireless communication capabilities to conduct communications with IPG 150.

Controller device 160 preferably provides one or more user interfaces to allow the user to operate pulse generator 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc. In the methods and systems described herein, parameters may include, for example, a number of pulses in a burst (e.g., 3, 4, or 5 pulses per burst), an intra-burst frequency (e.g., 130 Hz), an inter-burst frequency (e.g., 3-20 Hz), and a delay between a first and second burst (e.g., less than 1 millisecond (ms)).

IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 2001/093953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference. Example commercially available neurostimulation systems include the EON MINI™ pulse generator and RAPID PROGRAMMER™ device from St. Jude Medical, Inc. (Plano, Tex.).

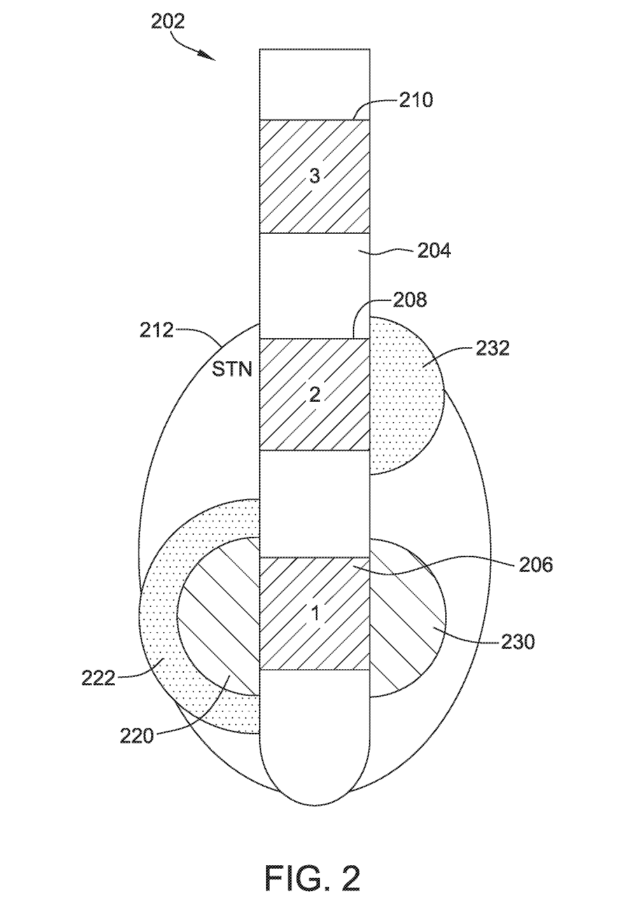

FIG. 2 is a schematic view of a portion of a stimulation lead 202 that may be used, for example, with neurostimulation system 100 (shown in FIG. 1). In this embodiment, stimulation lead 202 includes a distal segment 204 that includes a first contact 206, a second contact 208, and a third contact 210. Alternatively, distal segment 204 may include any suitable number of contacts, or electrodes. Contacts 206, 208, and 210 may be, for example, ring electrodes or segmented electrodes.

As shown in FIG. 2, in this embodiment, stimulation lead 202 is a DBS lead implanted into a patient's brain. Specifically, first and second contacts 206 and 208 are positioned within a subthalamic nucleus (STN) 212 of the patient, while third contact 210 is positioned outside of STN 212.

As will be explained in detail, stimulation lead 202 delivers neurostimulation to activate different populations of neurons within a small region of the brain (e.g., STN 212) in an uncoordinated sequence. Using only one or two contacts (e.g., first and/or second contacts 206 and 208), targeted stimulation is delivered to a very specific region, while still disrupting pathologic oscillation.

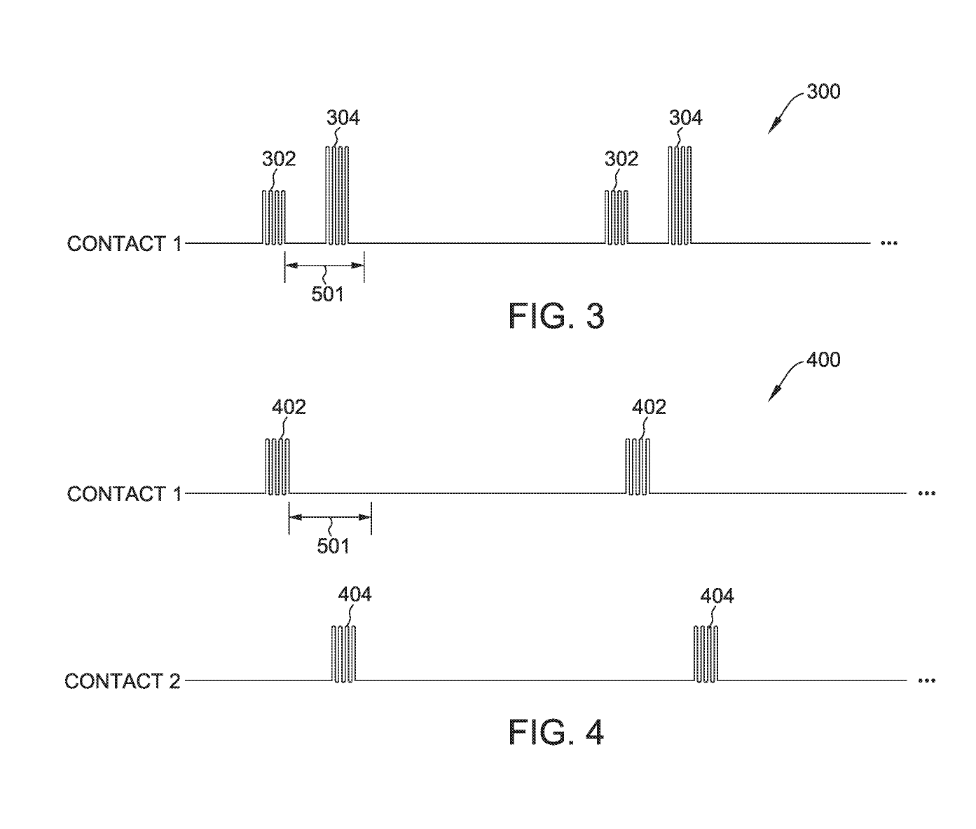

FIGS. 3 and 4 are diagrams 300 and 400 of example stimulation patterns that may be applied over time using stimulation lead 202. Diagram 300 shows one example stimulation pattern in accordance with a first embodiment, and diagram 400 shows one example stimulation pattern in accordance with a second embodiment.

In the first embodiment, stimulation is delivered using a contact, such as first contact 206, but at different amplitudes. In the example shown in FIG. 3 a first burst 302 of stimulation is provided at a relatively low amplitude, activating only neurons that are relatively close to first contact 206. Subsequently, a second burst 304 of stimulation is provided at a higher amplitude, activating neurons within a larger region. First and second bursts 302 and 304 are then repeated, as shown in FIG. 3. The amplitudes of first and second bursts 302 and 304 may be selected randomly or psuedorandomly from two or more fixed values, or may be selected randomly or psuedorandomly from within predetermined ranges.

For example, first burst 302 may have an amplitude in a range from approximately 1 to 4 milliamps (mA), and second burst 304 may have an amplitude in a range from approximately 0.25 to 2 times larger or smaller than the amplitude of first burst 302.

In some embodiments, a pulse width of first and second bursts 302 and 304 is modulated instead of amplitude. In FIG. 2, a first tissue volume 220 activated in response to the stimulation delivered by first burst 302 is shown schematically, and a second tissue volume 222 activated in response to the stimulation delivered by second burst 304 is shown schematically.

In the second embodiment, stimulation is delivered using two contacts, such as first and second contacts 206 and 208, in a random pattern, in a pseudo-random pattern, in an in-sequence pattern, or substantially simultaneously. To activate three different neural populations, stimulation may be delivered at first contact 206, at second contact 208, or at both first and second contacts 206 and 208. As with the first embodiment, the amplitude and/or pulse width of each burst of stimulation may be modulated.

In the example shown in FIG. 4, a first burst 402 of stimulation is provided at a relatively low amplitude by first contact 206, activating only neurons that are relatively close to first contact 206. Subsequently, a second burst 404 of stimulation is provided at substantially the same amplitude by second contact 208, activating only neurons that are relatively close to second contact 208. Alternatively, second burst 404 may have a different amplitude, pulse width, etc. as compared to first burst 402.

First and second bursts 402 and 404 are then repeated, as shown in FIG. 4. In FIG. 2, a first tissue volume 230 activated in response to the stimulation delivered by first burst 402 is shown schematically, and a second tissue volume 232 activated in response to the stimulation delivered by second burst 404 is shown schematically.

In some embodiments, stimulation may be applied simultaneously at first and second contacts 206 and 208, using a bipolar configuration or a dual monopolar configuration, to activate a neural population distinct from that which would be activated by first contact 206 or second contact 208 alone. This simultaneous stimulation may be applied within a refractory period of first burst 402 or second burst 404, whichever occurs later. Alternatively, first burst 402 could be applied, followed by simultaneous stimulation (i.e., using both first and second contacts 206 and 208) within the refractory period of first burst 402, followed by application of second burst 404 within the refractory period of the simultaneous stimulation. Those of skill in the art will appreciate that many other different combinations of pulse timing are within the scope of the disclosure.

In both diagram 300 and diagram 400, second bursts 304 and 404 are delivered within a neuronal refractory period 501 after respective first bursts 302 and 402. That is, following excitation, neurons excited with a first burst are unable to be excited again until after neuronal refractory period 501 passes. Neuronal refractory period 501 has a duration of approximately 1 millisecond (ms). By delivering second bursts 304 and 404 within neuronal refractory period 501, a distinct neural population (relative to the neural population targeted by first bursts 302 and 402) is targeted with second bursts 304 and 404. For example, a neural population within first tissue volume 220 will not be activated by second burst 304. Rather, second burst 304 will activate the neural population in the “shell” of second tissue volume 222 that surrounds first tissue volume 220.

In some embodiments, stimulation lead 202 includes bipolar recording electrodes that are used to sense neuronal oscillation patterns and identify perturbation generated by the burst patterns applied using first and/or second contacts 206 and 208. For example, the bipolar recording electrodes may record local field potentials, phase-amplitude coupling signals, and/or phase-phase coupling signals.

Notably, signals recorded by bipolar recording electrodes may be used in a feedback scheme, where the burst patterns delivered by first and/or second contacts 206 and 208 are adjusted based on detected changes in neuronal activity. The bipolar recording electrodes may be the same as those used to deliver stimulation (i.e., first and second contacts 206 and 208), or may be distinct, non-stimulating electrodes. For example, stimulation lead 202 may include a set of relatively closely spaced bipolar electrodes (not shown) for dedicated recording, the set of bipolar electrodes positioned near the first and second contacts 206 and 208. Alternatively, a far field sensing system could be implemented by altering stimulation lead 202 to enable recording by a first electrode within the implanted brain target, and by a second electrode that is located relatively remotely from the implanted brain target (e.g., in a subdural position just below the cortical surface).

Notably, the systems and methods described herein may be implemented using a stimulation lead having ring and/or segmented electrodes. The term “segmented electrode” is distinguishable from the term “ring electrode.” As used herein, the term “segmented electrode” refers to an electrode of a group of electrodes that are positioned at the same longitudinal location along the longitudinal axis of a lead and that are angularly positioned about the longitudinal axis so they do not overlap and are electrically isolated from one another. In contrast, a “ring electrode” is an electrode that extends about the entire circumference of the stimulation lead.

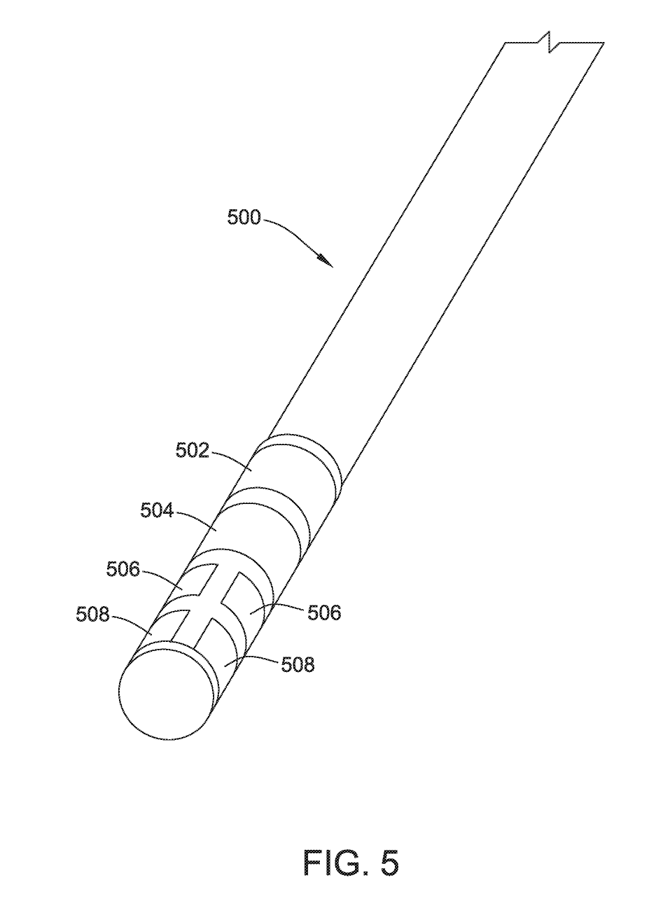

For example, FIG. 5 is a perspective view of a stimulation lead 500 that may be used to implement the systems and methods described herein. Stimulation lead 500 includes a first ring electrode 502 and a second ring electrode 504. Stimulation lead 500 further includes first segmented electrodes 506 located at a first longitudinal position, and second segmented electrodes 508 located at a second longitudinal position. In this embodiment, stimulation lead 500 includes three first segmented electrodes 506 and three second segmented electrodes 508. Alternatively, stimulation lead 500 may include any suitable number of ring electrodes and segmented electrodes (including none). Further, those of skill in the art will appreciate the both ring and segmented electrodes may be used to implement the systems and methods described herein.

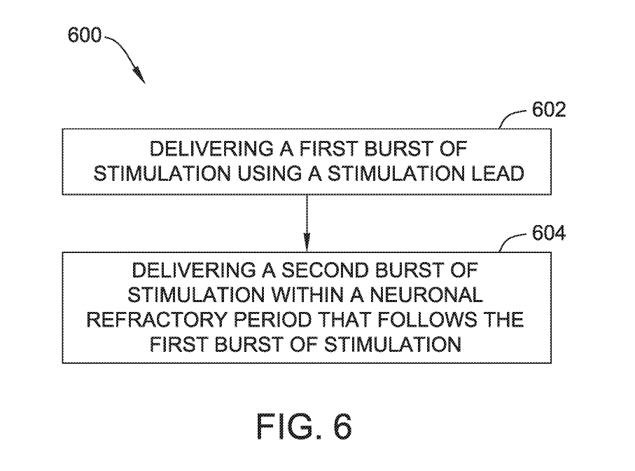

FIG. 6 is a flow chart of one embodiment of a method 600 for applying stimulation to disrupt neuronal oscillations in a patient. Method 600 includes delivering 602 a first burst of stimulation using a stimulation lead. Method 600 further includes delivering 604 a second burst of stimulation within a neuronal refractory period that follows the first burst of stimulation.

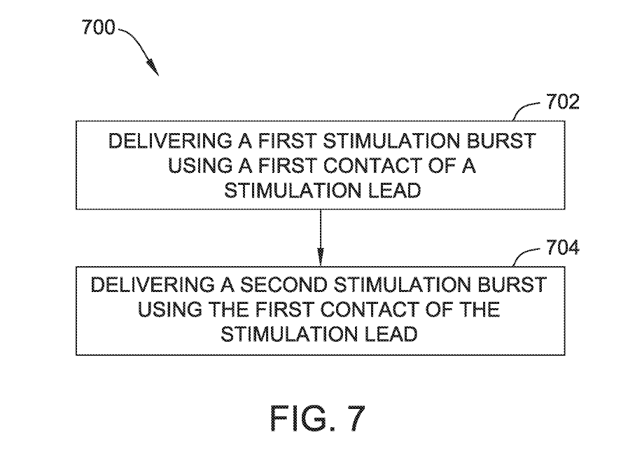

FIG. 7 is a flow chart of one embodiment of a method 700 for delivering first and second bursts of stimulation to disrupt neuronal oscillations in a patient. Method 700 may be used to implement, for example, delivering 602 and delivering 604 (both shown in FIG. 6). Method 700 includes delivering 702 a first stimulation burst using a first contact of a stimulation lead. Method 700 further includes delivering 704 a second stimulation burst using the first contact of the stimulation lead.

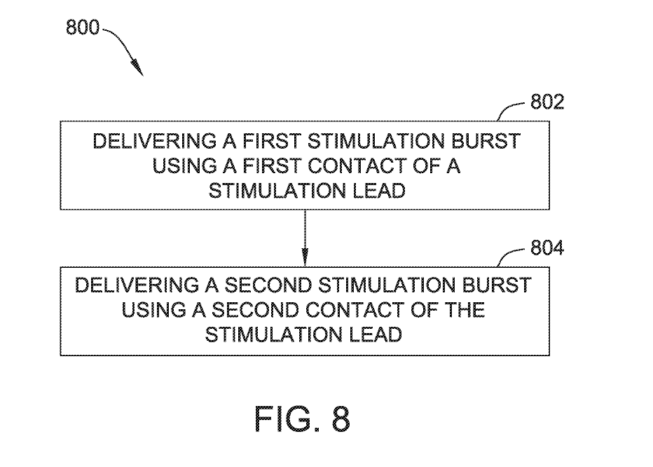

FIG. 8 is a flow chart of one embodiment of a method 800 for delivering first and second bursts of stimulation to disrupt neuronal oscillations in a patient. Method 800 may be used to implement, for example, delivering 602 and delivering 604 (both shown in FIG. 6). Method 800 includes delivering 802 a first stimulation burst using a first contact of a stimulation lead. Method 800 further includes delivering 804 a second stimulation burst using a second contact of the stimulation lead.

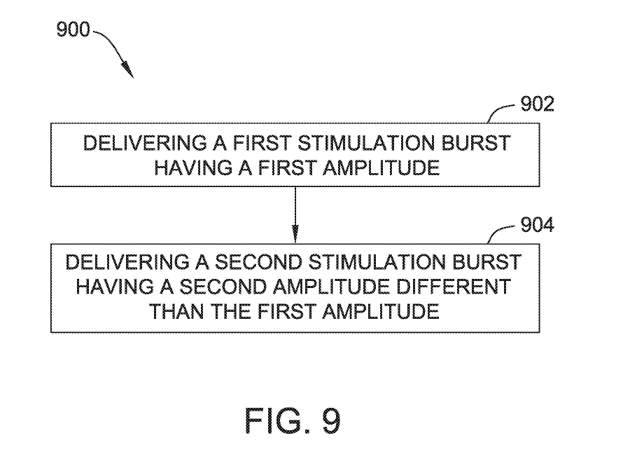

FIG. 9 is a flow chart of one embodiment of a method 900 for delivering first and second bursts of stimulation to disrupt neuronal oscillations in a patient. Method 900 may be used to implement, for example, delivering 602 and delivering 604 (both shown in FIG. 6). Method 900 includes delivering 902 a first stimulation burst having a first amplitude. Method 900 further includes delivering 904 a second stimulation burst having a second amplitude different than the first amplitude.

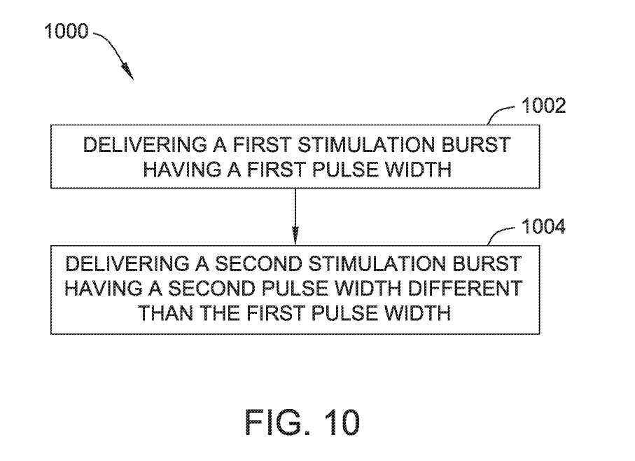

FIG. 10 is a flow chart of one embodiment of a method 1000 for delivering first and second bursts of stimulation to disrupt neuronal oscillations in a patient. Method 1000 may be used to implement, for example, delivering 602 and delivering 604 (both shown in FIG. 6). Method 1000 includes delivering 1002 a first stimulation burst having a first pulse width. Method 1000 further includes delivering 1004 a second stimulation burst having a second pulse width different than the first pulse width.

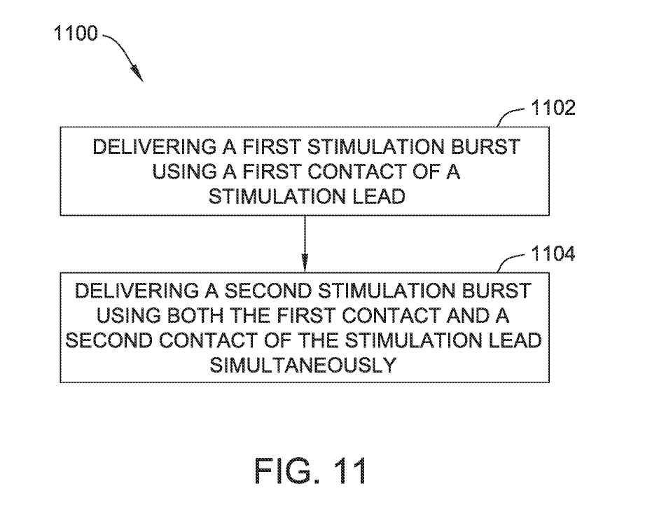

FIG. 11 is a flow chart of one embodiment of a method 1100 for delivering first and second bursts of stimulation to disrupt neuronal oscillations in a patient. Method 1100 may be used to implement, for example, delivering 602 and delivering 604 (both shown in FIG. 6). Method 1100 includes delivering 1102 a first stimulation burst using a first contact of a stimulation lead. Method 1100 further includes delivering 1104 a second stimulation burst using both the first contact and a second contact of the stimulation lead simultaneously.

Notably, at least one difference between the systems and methods described herein and a coordinated reset stimulation pattern is that the systems and methods described herein utilize two or fewer contacts to disrupt pathological activity, instead of requiring three contacts to deliver stimulation. Further, in the systems and methods described herein, after delivering a first burst of stimulation, a second burst of stimulation is delivered within a neuronal refractory period.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A neurostimulation system for disrupting neuronal oscillations, the neurostimulation system comprising:
a stimulation lead comprising at least one contact; and
an implantable pulse generator (IPG) communicatively coupled to the stimulation lead and configured to cause stimulation to be applied to a patient using no more than two contacts of the stimulation lead by:
causing a first burst of stimulation to be delivered; and
causing a second burst of stimulation to be delivered within a neuronal refractory period that follows the first burst of stimulation.

2. The neurostimulation system of claim 1, wherein to cause stimulation to be applied, the IPG is configured to:
cause the first burst of stimulation to be delivered by a first contact of the stimulation lead; and
cause the second burst of stimulation to be delivered by the first contact.

3. The neurostimulation system of claim 1, wherein to cause stimulation to be applied, the IPG is configured to:
cause the first burst of stimulation to be delivered by a first contact of the stimulation lead; and
cause the second burst of stimulation to be delivered by a second contact of the stimulation lead.

4. The neurostimulation system of claim 1, wherein the first burst of stimulation has a different amplitude than the second burst of stimulation.

5. The neurostimulation system of claim 1, wherein the first burst of stimulation has a different pulse width than the second burst of stimulation.

6. The neurostimulation system of claim 1, wherein to cause stimulation to be applied, the IPG is configured to cause stimulation to be applied to at least one of a subthalamic nucleus, a globus pallidus interna, or a thalamus of the patient.

7. The neurostimulation system of claim 1, wherein the stimulation lead further comprises a plurality of recording electrodes.

8. The neurostimulation system of claim 1, wherein the at least one contact comprises a plurality of segmented electrodes.

9. An implantable pulse generator (IPG) for use with a neurostimulation system for disrupting neuronal oscillations, the IPG comprising:
a memory device; and
a controller communicatively coupled to the memory device, the controller configured to cause stimulation to be applied to a patient using no more than two contacts of a stimulation lead coupled to the IPG by:
causing a first burst of stimulation to be delivered; and
causing a second burst of stimulation to be delivered within a neuronal refractory period that follows the first burst of stimulation.

10. The IPG of claim 9, wherein to cause stimulation to be applied, the controller is configured to:
cause the first burst of stimulation to be delivered by a first contact of the stimulation lead; and
cause the second burst of stimulation to be delivered by the first contact.

11. The IPG of claim 9, wherein to cause stimulation to be applied, the controller is configured to:
cause the first burst of stimulation to be delivered by a first contact of the stimulation lead; and
cause the second burst of stimulation to be delivered by a second contact of the stimulation lead.

12. The IPG of claim 9, wherein the first burst of stimulation has a different amplitude than the second burst of stimulation.

13. The IPG of claim 9, wherein the first burst of stimulation has a different pulse width than the second burst of stimulation.

14. The IPG of claim 9, wherein to cause stimulation to be applied, the controller is configured to cause stimulation to be applied to at least one of a subthalamic nucleus, a globus pallidus interna, and a thalamus of the patient.

15. A method of applying stimulation using no more than two contacts of a stimulation lead to disrupt neuronal oscillations in a patient, the method comprising:
delivering a first burst of stimulation using the stimulation lead; and
delivering a second burst of stimulation within a neuronal refractory period that follows the first burst of stimulation.

16. The method of claim 15, wherein delivering a first burst comprises delivering the first burst using a first contact of the stimulation lead, and wherein delivering a second burst comprises delivering the second burst using the first contact.

17. The method of claim 15, wherein delivering a first burst comprises delivering the first burst using a first contact of the stimulation lead, and wherein delivering a second burst comprises delivering the second burst using a second contact of the stimulation lead.

18. The method of claim 15, wherein delivering a first burst comprises delivering a first burst having a first amplitude, and wherein delivering a second burst comprises delivering a second burst having a second amplitude that is different than the first amplitude.

19. The method of claim 15, wherein delivering a first burst comprises delivering a first burst having a first pulse width, and wherein delivering a second burst comprises delivering a second burst having a second pulse width that is different than the first pulse width.

20. The method of claim 15, wherein delivering a first burst comprises delivering the first burst using a first contact of the stimulation lead, and wherein delivering a second burst comprises delivering the second burst using both the first contact and a second contact of the stimulation lead simultaneously.
